(19) United States
(12) Patent Application Publication
(10) Pub. No.: US 2006/0051402 A1
(43) Pub. Date: Mar. 9, 2006

(54) Splinting orthopedic and rehabilitative product

(75) Inventors: Ronald F. Bogardus, Hollis, NH (US); John J. Duren, Kewaskum, WI (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: AbilityOne Corporation, Bolingbrook, IL (US)

(21) Appl. No.: 10/936,742

(22) Filed: Sep. 8, 2004

(51) Int. Cl. A61L 15/16

(52) U.S. Cl. 424/444; 424/405; 424/618

(57) ABSTRACT

The invention provides a splinting orthopedic or a rehabilitative product that has an increased resistance to odor and/or microbial growth. More particularly, the invention provides for a moldable splinting orthopedic or a rehabilitative product comprising an antibiotic agent and low temperature thermoplastic polymer.

### FIELD OF THE INVENTION

The invention relates to moldable orthopedic splints and rehabilitative products that have an increased resistance to odor and/or microbial growth.

### BACKGROUND OF THE INVENTION

Orthopedic splints provide a temporary means for many therapeutic uses including mechanical support, prevention of deformity, and maintenance of corrected posture. The materials commonly used for splints suffer from a number of disadvantages. Plaster has a low strength-to-weight ratio, heavy weight, bulk, and tendency to disintegrate in water. In addition, plaster casts do not allow for air circulation, which traps moisture on the cast and skin leading to skin irritation or infection, odor, and general discomfort for the patient. The development of polymer casting materials, such as fiberglass, helped to eliminate many of the disadvantages of plaster casts, particularly weight and air permeability. Nevertheless, these materials are less compressible than plaster, can interfere with X-ray images, and are expensive.

Thermoplastic materials possess desirable physical characteristics for splinting applications, such as being easy to handle, conformable, moldable, lightweight, economic, convenient, and strong. A filled thermoplastic crystalline solid polyurethane cast material is disclosed in U.S. Pat. No. 4,473,671 (Green). In use, this cast material is warmed to a sufficiently high temperature to cause the polymer to become soft enough to deform, and is shaped as desired. Once cooled to ambient temperature the thermoplastic cast hardens and holds its shape. U.S. Pat. No. 4,454,873 (Laufenberg) discloses an orthopedic cast material comprising a thermoplastic material and a coating of polyethylene oxide. The coating is included to prevent adherence of the casting material to itself when heated.

All of the above materials suffer from a common disadvantage in that they are prone to odor and microbial growth. Such growth can be a cause of odor, but can also cause more serious problems, such as skin irritation or infection, or simply a lack of general hygiene. Providing a thermoplastic casting material that has an increased resistance to microbial growth would help to eliminate odor from casts, provide a more sterile surface-to-skin contact, and prevent degradation, discoloration, and/or deformation of the casting material due to the presence of microbes. Thus, microbial growth that causes skin irritation, splint deformation, splint degradation, splint discoloration, malodors, and skin infection that occur during use of casts and splints made with known casting and splinting materials could be eliminated.

The use of antimicrobial agents for treating medical devices that are implanted or inserted into the body is known in the art. For example, U.S. Pat. No. 6,589,591 relates to a method for treating non-metallic indwelling or implanted medical devices with a composition that includes an antimicrobial agent, an acid solution, and glycerol. (See also U.S. Pat. No. 4,895,566; U.S. Pat. No. 4,917,686; U.S. Pat. No. 4,107,121; U.S. Pat. No. 5,013,306; U.S. Pat. No. 4,952,419; U.S. Pat. No. 4,442,133) These patents disclose medical articles such as catheters, drain tubes, stents, stints, joint replacements, valves, and implants which are all intended for use on the interior of the body. These patents do not discuss the utility of a casting material comprising an antibiotic in an amount sufficient to inhibit microbial growth.

As evident from above, even though casting materials have continued to improve, there remains a need in the art for a cast material that has the advantages of known cast materials, but with an increased resistance to odor and microbial growth. The invention described herein provides improved cast materials that are resistant to odor and/or microbial growth.

### SUMMARY OF THE INVENTION

In one aspect, the invention relates to an external casting material comprising a low temperature thermoplastic polymer, wherein the low temperature thermoplastic polymer comprises an amount of an antibiotic effective to inhibit microbial growth during the length of use of the casting material.

In another aspect, the invention relates to a method for manufacturing an external casting material comprising (a) forming a low temperature thermoplastic polymer into a sheet, a preformed case, or a particular shape; and (b) contacting the low temperature thermoplastic polymer formed in (a) with an amount of a composition comprising an antibiotic effective to inhibit microbial growth.

In a further aspect, the invention relates to a method for manufacturing an external casting material comprising (a) contacting a low temperature thermoplastic polymer with an amount of an antibiotic effective to inhibit microbial growth; (b) mixing the low temperature thermoplastic polymer and antibiotic for a time sufficient to evenly distribute the antibiotic throughout the low temperature thermoplastic polymer; and (c) forming the mixed low temperature thermoplastic polymer and antibiotic to form a sheet, a preformed case, or a particular shape.

In another aspect, the invention relates to kits comprising the external casting material of the invention along with instructions for use.

Further aspects and advantages of the invention will become apparent from the following detailed description of the invention.

### DETAILED DESCRIPTION OF THE INVENTION

### DEFINITIONS

Unless defined otherwise, all scientific and technical terms used herein have the same meaning as commonly understood by one of skill in the art to which this invention belongs. All patents and publications referred to herein are hereby incorporated by reference for all purposes.

As used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

By “antibiotic” is meant any class of compound and/or agent that can inhibit or prevent growth of a microorganism. Antibiotics include the non-limiting examples of biocides, antimicrobial compounds, such as antibacterials, antivirals, and antifungals; pesticides; mildewcides; and dessicants. It should be understood that “antibiotic” can refer to one or more than one antibiotic compound and/or agent, so that a casting material comprising an antibiotic can encompass a single antibiotic, or a combination of two or more antibiotics.

“Low temperature thermoplastic,” “low temperature thermoplastic resin,” or “low temperature thermoplastic polymer” is taken to mean any polymer material that has a melting temperature of 48° C. to 80° C. and hardens when cooled. “Melting temperature,” as used herein, means the temperature at which the thermoplastic polymer begins to soften and becomes moldable. Thermoplastic polymers are known in the art and are used in casting materials and as non-metallic medical devices. Any known casting material comprising a low temperature thermoplastic polymer can be used in the instant invention, such as the non-limiting examples of polycapralactone, transpolyisoprene rubber, polycapralactone-derived materials, and materials containing as a component polycapralactone and/or transpolyisoprene rubber (“blends”). It should be recognized that elastomers that fall within the above-recited melting temperature range are also included within the meaning of low temperature thermoplastic.

“Growth inhibition” or “inhibition of growth” both refer to inhibition of microbial growth. As used herein, inhibition can mean preventing, slowing, or inhibiting growth. For example, inhibition of growth can mean a complete inhibition of microbial growth, preventing further microbial growth, or slowing the rate of microbial growth. Inhibition is measured relative to casting materials or low temperature thermoplastic polymers that do not comprise an antibiotic compound and/or agent. Thus, inhibition of growth means no growth, or any measurable amount of microbial growth in or on a casting material comprising an antibiotic agent that is less than the amount of microbial growth in or on a casting material that does not comprise an antibiotic agent, under the same or similar environmental conditions. Inhibition should also be interpreted to encompass casting materials that can delay onset of microbial growth for a longer period of time than other casting materials, whether during use or storage. It follows from this definition of growth inhibition that the term “an amount of an antibiotic effective to inhibit microbial growth” means at least the minimum weight percentage or concentration of an antibiotic agent/compound that inhibits growth.

“Microbe” or “microbial” refers to any organism classified or commonly known as a microbe, for example, bacteria (e.g., Gram-positive, Gram-negative), viruses, fungi, and yeast. In addition, for purposes of this invention, microbe can also be taken to mean pestilent organisms such as insects, including mites, ticks, fleas, chiggers, flies, and the like.

In one aspect, the invention relates to an external casting material comprising a low temperature thermoplastic polymer, wherein the low temperature thermoplastic polymer comprises an amount of an antibiotic effective to inhibit microbial growth during the length of use of the casting material.

The casting material can be used to form any type of splint, cast, support, drape, or corset. The casting material can be produced in sheets, extruded to form particular shapes, or be cut into any desired preformed shape. The antibiotic agent can be incorporated into (as an additive) or onto (as a coating) the low temperature thermoplastic polymer by any method known in the art, as long as the resulting product retains the favorable physical characteristics of the thermoplastic polymer, and the antibiotic retains sufficient activity to inhibit microbial growth. The casting materials of the invention can be used for any mammalian subject or patient in need of treatment. A subject or patient, as used herein, can be any mammal, but is preferably a human subject.

Antibiotic/Antimicrobial Agent

The antibiotic agents of the invention can be used in the neutral or the salt form, or a mixture of those two forms. Any antibiotic agent can be used in the invention as long as it is present in an amount sufficient to inhibit microbial growth. The considerations and selection of the particular antibiotic agent for a specific application is familiar to those of skill in the art. Briefly, considerations include such non-limiting factors as antibiotic agent solubility, effective concentration ranges, activity, stability, and/or potential for irritation or allergic reaction. Potential problems such as irritation or allergic reaction can be controlled by the particular formulation of the antibiotic agent, or by including other components that alleviate the irritation or allergic reaction. For example, other agents that help control or relieve irritation or allergic reaction, such as, for example, skin conditioning agents, (e.g., aloe, lanolin, moisturizer, etc.); desiccants and/or absorbents (e.g., talc), can be incorporated into or onto any one or more of the low temperature thermoplastic resin, the coating composition, or the antibiotic agent, depending on the particular components. Further, the method of applying the antibiotic agent can help to reduce or eliminate irritation or allergic reaction by, for example, limiting the amount of antibiotic agent transferred and/or exposed to the skin. Further, other purely aesthetic components can be included in these casting materials and splints, such as dyes, graphic designs, and/or fragrance.

As mentioned above, the selection of an antibiotic is based on a number of requirements. These include the compatibility of the antibiotic with the protective coating material (e.g., solubility and stability); its ability to integrate or coat the low temperature thermoplastic polymer; its effective amount that will inhibit the growth of microorganisms during the storage and/or use of the coated thermoplastic resin; the duration of its efficacy; its stability to moisture (including water and sweat), pH, and air; and its non-irritation potential and sensitization to mammalian skin during and after use of the low temperature thermoplastic products in the prescribed manner, as well as other factors known to those of skill in the art.

Candidate antibiotics can be selected and initially screened based on information from technical data and published reports and studies that demonstrate that the antibiotic(s) has the required physical and chemical attributes for the particular application. For the purposes of the invention, the antibiotics comprise agents that do not degrade (i.e., they retain antibiotic efficacy) during storage of the coated thermoplastic products prior to use, and do not degrade the performance of the deposited protective coating on the thermoplastic sheet either by generating surface imperfections, discoloration, malodors or loss of adhesion to the thermoplastic sheet during or after use. That is, the antibiotic agents useful in the invention allow the thermoplastic polymer to function as intended. Nevertheless, depending on the antibiotic agent, particular storage conditions can be recommended, such as shielding from light or heat, which will help to prolong the storage life of the product.

Antibiotic agents (i.e., antibiotics, antimicrobials, antibacterials, biocides, fungicides, pesticides, etc.) consist of many structurally different substances including, but not limited to, parabens, halogenated compounds, tin complexes, phenol derivatives, arsine derivatives, sulphones, N-substituted thiocarbamates, organic and inorganic silver salts, as well as numerous other compounds which are well known in the art (Coatings World, May 2004, pp. 40-42; Paint and Coating Industry, July 2003, p. 64; Paint and Coating Industry, December 2002, p. 63).

In one aspect, the external casting materials of the invention comprise amounts of antibiotic agent that are effective to inhibit proliferation or growth of microbes, typically from about 50 ppm to about 5,000 ppm (0.005%-0.500%). The effective amounts will vary according to particular parameters including, for example, the chemical and physical characteristics of the casting material, the location and intended use of the casting material, and the chemical and physical characteristics of the antibiotic. If a combination of two or more antibiotics is used with the invention, each can be present in an amount that is not ordinarily effective to inhibit growth, but together result in an amount that function to inhibit growth. Such a combination can exhibit either an additive or a synergistic effect. The most important consideration in selecting an antibiotic agent is that it is able to inhibit microbial growth when used in the invention. All of these considerations are well within the knowledge of those skilled in the art.

In an embodiment of this aspect, the effective amount for inhibiting microbial growth is from about 75 ppm to about 750 ppm (0.0075%-0.075%).

In a preferred embodiment of this aspect, the effective amount for inhibiting microbial growth is from about 50 to about 350 ppm (0.005%-0.035%).

In another aspect, the external casting materials of the invention comprise amounts of antibiotic agent that are effective to inhibit odor resulting from the use of the external casting materials, typically from about 50 ppm to about 5,000 ppm (0.005%-0.500%).

In an embodiment of this aspect, the effective amount for inhibiting odor is from about 75 ppm to about 750 ppm (0.0075%-0.075%).

In a preferred embodiment of this aspect, the effective amount for inhibiting odor is from about 50 to about 350 ppm (0.005%-0.035%).

In another embodiment the antibiotic is selected from triclosan, 2,4,4′-trichloro-2′-hydroxy-diphenylether (e.g., Irgasan, (Brenntag, N.V.) and Microban®), organo-silver compounds (e.g., Sanitized® Silver (Sanitized A.G.), and AlphaSan®, (Milliken Chemical), 10-10′-oxybisphenoxy arsine (e.g., Vinyzene™ (Rohm & Haas)), or 2-n-octyl-4-isothiazolin-3-one (Rohm & Haas). In a preferred embodiment the antibiotic is 2-n-octyl-4-isothiazolin-3-one (OIT). This antibiotic is particularly preferred because it has a history of extensive use in cosmetics, household paints, adhesives, soaps and detergents, and coatings. Other such specific antibiotics useful in the invention are known in the art.

Low Temperature Thermoplastic Polymer

Any type of low temperature thermoplastic polymer can be used with the invention. (See, generally, U.S. Pat. Nos. 6,107,366; 6,034,205; 5,763,075; 5,104,938; 5,055,525; 4,313,873 incorporated herein by reference). A preferred type of low temperature thermoplastic polymer is one that is currently known or used in casting materials. Additionally, elastomers can be used instead of, or in combination with, the low temperature thermoplastic polymer (for example, elastomers manufactured by DuPont Dow). While elastomers and thermoplastics typically can be distinguished by melting temperature (or glass transition temperature), elastomers of the invention are characterized in that they have a melting temperature falling within the range of the low temperature thermoplastic polymers of the invention. Thus, they provide for structural stability in the casting material. The most important consideration in selecting a low temperature thermoplastic polymer (or elastomer) is that it retains its melting temperature, moldability/flexibility, and strength when hardened, with addition of the antibiotic agent.

The low temperature thermoplastic polymer has a typical setting time, for example, from about 3 to about 12 minutes. One of skill in the art will be able to consider both the temperature and the setting time of the casting material and begin application of the casting material to the subject or patient so that the material retains sufficient flexibility and moldability, without the risk of burning or causing general discomfort to the patient or subject.

The low temperature thermoplastic polymer can stretch. The amount of stretch can vary widely, typically from about 10 to about 300%. This characteristic is most important in the embodiment of the invention comprising an antibiotic coating to a low temperature thermoplastic polymer, whether in a sheet or preformed cast or shape. In this embodiment, the amount of stretch should be adequate to allow for adequate formability, flexibility, and/or moldability, but should not exceed an amount that could cause the coating to crack, section, or separate. In most circumstances the physician, nurse, technician, or other person applying the external casting material can control the amount of stretch during the application and molding process.

The low temperature thermoplastic polymer can include, as noted above, other agents such as skin conditioning agents, (e.g., aloe, lanolin, etc.); desiccants and/or absorbents, (e.g., talc); dyes; and/or fragrance that can help to control or relieve irritation or allergic reaction, or are included for aesthetic reasons.

Typically, the low temperature thermoplastic polymer has an amount of tack, such that it is able to stick to itself during application. For particular applications a certain amount of tackiness is desirable, however too much tackiness could cause discomfort for the subject or patient during use and/or removal. In certain embodiments the external casting material can comprise a coating that helps to control the amount of tack to a desired level. This coating can further comprise an antibiotic in an amount effective to reduce or inhibit microbial growth. In another embodiment, the coating can be removed selectively, either physically or chemically, to reduce or increase the tack or antibiotic activity, depending on the composition of the coating and casting material.

In an embodiment of this aspect, the low temperature thermoplastic polymer has a melt temperature of about 48° C. to about 80° C.

In a preferred embodiment, the low temperature thermoplastic polymer is polycapralactone, compounds based on polycapralactone, blends of polycapralactone with other materials, or transpolyisoprene rubber.

Manufacturing

For certain manufacturing methods, it can be advantageous to form a solution of the antibiotic agent in an aqueous or an organic solvent or mixed solvent system. For purposes of this discussion “solution” should be taken to encompass its usual meaning, as well as to encompass dispersions, suspensions, colloids, and supersaturated solutions. Depending on the required concentration for the particular antibiotic agent to be used, appropriate salts or solvent systems can be used to adjust the required concentration of the solution. For other applications, it can be an advantage to add the antibiotic agent to the low temperature thermoplastic polymer during a mixing stage prior to extrusion or formation of the casting material, in order to disperse the antibiotic agent evenly throughout the polymer.

The antibiotic agent can be incorporated on or in the low temperature thermoplastic polymer in any way known to those of skill in the art, wherein the activity of the antibiotic agent and the dynamic physical characteristics of the polymer are maintained. Typically, the antibiotic is applied to the low temperature thermoplastic polymer material in two ways, although these should not be considered as limiting the invention. First, a coating comprising at least one antibiotic agent can be applied to the material via dip coating, dry spray, wet spray, brushing or a wipe-on method, so as to deposit a film thickness of between 0.0005-0.0050 inches, after air-drying at temperatures between 20-40° C. The application process for coating deposition can readily be adjusted in respects to drying rates and coating thickness desired by those skilled in the art of coating application.

Alternatively, the antibiotic agent can be combined with the base material during the processing stage. This processing can be done via compounding or as an additive included when processing the material. The method of incorporating the antibiotic agent can be selected based on a variety of factors, including the solubility or the required concentration of antibiotic agent, the particular low temperature thermoplastic polymer being used, and the type of external casting material to be formed, among other considerations known to those of skill in the art. For example, if the external casting material includes perforations (to aid in air circulation, for example) compounding the antibiotic agent with the polymer during the processing stage would be advantageous, since the coating application could block the perforations, or could fail to adequately coat the area immediately surrounding the perforations, thus potentially failing to inhibit microbial growth or prevent odor.

In an aspect, the invention relates to a method for manufacturing an external casting material comprising (a) forming a low temperature thermoplastic polymer into a sheet, preformed cast, or particular shape; and (b) contacting the low temperature thermoplastic polymer from (a) with an amount of a composition comprising an antibiotic effective to inhibit microbial growth.

In an embodiment of this aspect, the thermoplastic resin is coated with a composition comprising a water-resistant organic polymer and an amount of an antibiotic compound effective to inhibit microbial growth on its surface. For such coating applications, the thermoplastic resin can be in sheet form of varying dimensions, particular shapes, or in preformed orthopedic casts, splints, devices, and rehabilitative products. In one embodiment, the coating provides a permanent, non-tacky surface on the thermoplastic resin substrate that prevents adherence to itself during the molding or forming process and when applied in an orthopedic manner to mammalian skin and hair. This can provide greater comfort for the subject during the application and use of the product. Conversely, when it is advantageous to have a tacky surface, the coated thermoplastic resin can be manipulated to have enough tack to adhere to itself. Typically, a portion of the coating can be removed by application of a solvent, for example an alcohol such as isopropanol, across its surface (e.g., wiping with a cloth or swab), or the coating can also be removed by mechanical abrasion. Following removal of the coating, the surface tack of the thermoplastic resin is reactivated by moderate heating (e.g., with hot air, water, etc.) thereby allowing for self-adherence during the application (e.g., orthopedic) process.

The coating can be composed of a single polymer, copolymer, polymer blend or alloy such as, for example, polyvinylpyrolidone, polyacrylate, polyvinylacetate or the like, or any combination thereof. The polymers can be dispersed or dissolved in organic solvents, water, a combination of organic solvents and/or water, or a reactive two-component, crosslinked composition or reactive hybrid.

In one embodiment the type of coating is a waterborne single-component polyurethane dispersion which coalesces to form a film during solvent/water evaporation. The chemistry of waterborne urethanes is well known, and they can be ionic or non-ionic in nature. See, e.g., U.S. Pat. No. 4,183,836 (Wolfe, Jr.). Waterborne urethanes, including anionic urethanes, are preferred as they typically exhibit a favorable combination of physical characteristics (e.g., tensile strength, tear resistance, abrasion resistance, and the like). The anionic urethanes are typically prepared by reacting an excess of a polyfunctional diisocyanate (e.g., toluene diisocyanate, isophorone diisocyanate, methylenebis (cyclohexyisocyanate), and the like) with a polyether- or saturated polyester glycol, and a dispersing diol (e.g., dimethylol-proprionic acid and the like) to form a pre-polymer that is dispersed in water in the presence of a tertiary amine. The resulting pre-polymer can be extended with water or polyamine. Typically, the preferred polyurethane dispersion has a pH of about 7-8, a solids content of 30-40%, a low solution viscosity, and excellent storage stability. Further components can be added to the coating depending on the specific characteristics for the particular application, including dispersing aids, flow aids, coalescing agents, associative thickeners, pigments, dyes, fragrance, and compatible antibiotics.

In one embodiment, the method of manufacture comprises forming the thermoplastic resin into a net shape via injection molding, extrusion, or compression molding. The net shape can be in the form of a sheet of any thickness or a dimensional shape. The shape can then be either coated with the antibiotic agent or the antibiotic agent can be added when the thermoplastic resin is formed into the sheet or final product. In a further preferred embodiment the method comprises forming a desired shape via injection molding or extrusion.

In a further aspect, the invention relates to a method for manufacturing an external casting material comprising (a) contacting a low temperature thermoplastic polymer with an amount of an antibiotic effective to inhibit microbial growth; (b) mixing the low temperature thermoplastic polymer and antibiotic for a time sufficient to evenly distribute the antibiotic throughout the low temperature thermoplastic polymer; and (c) forming the mixed low temperature thermoplastic polymer and antibiotic into the form of a sheet.

In another aspect, the invention also provides a kit comprising one or more sheets of casting material of the invention, and instructions for use. In one embodiment of this aspect, the one or more sheets of the casting material is prepared in a pre-formed shape or cast.

The following Examples serve merely to illustrate certain embodiments of the invention and should not be taken to limit the invention, as defined in the appended claims, in any way.

### EXAMPLES

### Example 1

### Selection of Antibiotic Compound(s)

A simple screening program was devised to determine the solution compatibility of antibiotic candidate compounds when dispersed in the protective coating. A known amount of a selected antibiotic, 2-n-octyl-4-isothiazolin-3-one (OIT) in this example, based on the antibiotic recommended minimum inhibitory concentration (MIC) level, is added to a stock solution of the coating base, which is, in this example, a polyurethane dispersion (Paint & Coatings Industry, July 2003, pp. 66-72). The resulting coating mixture, containing 0.03% (by weight) of a 45% solution of OIT, is placed in glass test tubes, sealed, and is subjected to 3× freeze-thaw cycles (from −20° C. to 40° C.). Thereafter, the mixture is observed for any indication of separation of components.

In addition to the freeze-thaw trials, viscosity determinations are conducted in order to observe changes in solution viscosities and the potential formation of gel particles over the course of a five day period at ambient (room) temperature. At the conclusion of the storage cycle several 6 mil (0.006 inches) wet film draw downs are deposited on the thermoplastic sheeting, air-dried at 25° C. and examined for film integrity, adhesion and appearance. Subsequent hot water immersion of the coated thermoplastic sheet and 180° mandrel bending are conducted to determine the effects of the antibiotic on the protective coating appearance and integrity. Standards using the polyurethane dispersion without antibiotic are used to establish a baseline point of reference for each of the measurements as well as viscosity, clarity, drying rate, film integrity, and adhesion.

### Example 2

### Preparation of an Antibiotic-Coated Thermoplastic Sheet

Test-coated thermoplastic sheet specimens were prepared by dip coating the thermoplastic sheet with the antibiotic coating (described above), in such a manner to obtain a final dry film thickness of approximately 0.001 to 0.003 in., at about 25° C.

### Example 3

### Antimicrobial Activity of Antibiotic-Coated Thermoplastic Sheet

The antibiotic coating for the thermoplastic sheet, as described in Examples 1 and 2 was cut into several 3 mm thick discs, each having a diameter of 9 mm. These discs were submitted to an independent test facility for a determination of antibiotic activity using antimicrobial screening tests.

The antibiotic testing assessed both bactericidal and fungicidal activity of the antibiotic coating. Antibacterial activity (against S. aureus) is observed as a ‘zone of inhibition’ and expressed as millimeters of inhibition of bacterial growth around each sample disc, as outlined in AATCC Test Method 147 (AATCC. Antibacterial activity assessment of textile materials: Parallel streak method. Research Triangle Park, N.C.: American Association of Textile Chemists and Colorists; AATCC Test Method 147-1988). Antifungal activity was measured using American Society for Testing and Materials (ASTM) Standard Test Method G-21-96 (“Standard Practice for Determining Resistance of Synthetic Polymeric Surfaces to Fungi”, 2002) and is expressed as percent growth over a given time. Mixed fungal groupings used for the test consisted of an equal number of spores of Aspergillus niger, Aureobasdiium pullans, Chaetomium globosum, Pencillium pinophilum, and Trichoderma virens.

The coating sample was able to inhibit bacterial growth in a zone of inhibition that extended 3 mm from the edge of the disc, over a 24 hr time period. The sample also completely inhibited fungal surface growth over a time period of 28 days (0% growth, measured at 7, 14, 21, and 28 days).

### Example 4

### Skin Irritation Potential of Antibiotic-Coated Thermoplastic Sheet

The antibiotic coating for the thermoplastic sheet, as described in Examples 1, 2, and 3 was cut into several 3 mm thick discs, each having a diameter of 9 mm. These discs were submitted to an independent test facility for a determination of skin irritation potential using patch tests.

A modified, shared panel Draize Assay was used to assess mammalian skin irritation potential of the antibiotic coating. The assay consists of an induction phase followed by a challenge phase. For the induction phase, test samples were applied to skin for a 48 hr. period, removed, and observed for signs of reaction or response. New test samples were then applied and the 48 hr. process repeated for ten cycles. After each 48 hr. cycle, any reactions and responses that subjects have to the test sample were recorded using a rating scale from 0-4, with 0 meaning no reaction and 4 meaning significant adverse skin reaction. This completed the induction phase portion of the test. A two week resting phase followed the induction phase and preceded the challenge phase. For the challenge phase, test samples were applied for 48 and 72 hr. periods and any reactions and responses noted. Under the conditions employed in the study, no evidence of skin irritation or sensitization was observed.

It is understood that the foregoing description is provided to illustrate particular embodiments of the instant invention, and should not be viewed as narrowing the scope or spirit of the invention as defined by the appended claims.

We claim:

1. An external casting material, comprising:
a low temperature thermoplastic polymer, wherein the low temperature thermoplastic polymer comprises an amount of an antibiotic effective to inhibit microbial growth during the length of use of the casting material.

2. The external casting material of claim 1, wherein the antibiotic is dispersed throughout the low temperature thermoplastic polymer.

3. The external casting material of claim 1, wherein the antibiotic is coated on the surface of the low temperature thermoplastic polymer.

4. The external casting material of claim 1, wherein the low temperature thermoplastic polymer is polycapralactone, polycapralactone blends, polycapralactone derivative, or transpolyisoprene rubber.

5. The external casting material of claim 3, wherein the antibiotic coated on the surface comprises a polyurethane dispersion.

6. The external casting material of claim 1, wherein the antibiotic is an antimicrobial or a pesticide.

7. The external casting material of claim 6, wherein the antibiotic is an antimicrobial.

8. The external casting material of claim 7, wherein the antimicrobial is triclosan, 2,4,4′-trichloro-2′-hydroxy-diphenylether, organo-silver compounds, 10-10′-oxybisphenoxy arsine, or 2-n-octyl-4-isothiazolin-3-one.

9. The external casting material of claim 6, wherein the antibiotic is a pesticide.

10. The external casting material of claim 9, wherein the pesticide comprises at least one compound effective to prevent ticks and fleas.

11. The external casting material of claim 1, wherein the external casting material comprises perforations.

12. The external casting material of claim 1, further comprising padding, gel, or cloth.

13. A method for manufacturing an external casting material comprising:
(a) forming a low temperature thermoplastic polymer into a sheet, preformed cast, or particular shape; and
(b) contacting the low temperature thermoplastic polymer formed in (a) with an amount of a composition comprising an antibiotic effective to inhibit microbial growth.

14. The method of claim 13, wherein the contacting step comprises coating the low temperature thermoplastic polymer sheet with the composition comprising an antibiotic.

15. The method of claim 14, wherein the composition is a liquid composition.

16. The method of claim 14, wherein the coating is performed by dip coating, dry spray, wet spray, or wipe-on application.

17. A method for manufacturing an external casting material comprising:
(a) contacting a low temperature thermoplastic polymer with an amount of an antibiotic effective to inhibit microbial growth;
(b) mixing the low temperature thermoplastic polymer and antibiotic from (a) for a time sufficient to distribute the antibiotic throughout the low temperature thermoplastic polymer;
(c) forming the low temperature thermoplastic polymer and antibiotic from (b) to form a sheet, preformed cast, or particular shape.

18. The method of claim 17, wherein the contacting step (a) comprises dry blending.

19. The method of claim 17, wherein the contacting step (a) comprises contacting a melt or a solution of the low temperature thermoplastic polymer with the antibiotic.

20. An external casting material, comprising:
a low temperature thermoplastic polymer, wherein the low temperature thermoplastic polymer comprises a polyurethane-based coating, wherein the polyurethane-based coating comprises an amount of an antibiotic effective to inhibit microbial growth, wherein the antibiotic comprises 2-n-octyl-4-isothiazolin-3-one, and wherein the low temperature thermoplastic polymer is a polycapralactone, a polycapralactone-based derivative, a polycapralactone blend, or transpolyisoprene rubber.
